(19) United States
(12) Patent Application Publication
(10) Pub. No.: US 2018/0126333 A1
(43) Pub. Date: May 10, 2018

(54) Device and a Method for Cleaning a Gas

(71) Applicant: Hermanus Johannus Theodorus Maria Taris, Den Andel (NL)

(72) Inventor: Hermanus Johannus Theodorus Maria Taris, Den Andel (NL)

(21) Appl. No.: 15/864,721

(22) Filed: Jan. 8, 2018

Related U.S. Application Data
(63) Continuation of application No. PCT/NL2016/050483, filed on Jul. 6, 2016.

(30) Foreign Application Priority Data
Jul. 6, 2015 (NL) 2015095

(51) Int. Cl. B01D 53/92; A61L 9/015; F23D 14/32; B01D 53/00; B01D 53/76; F01N 3/36

(52) U.S. Cl. CPC B01D 53/92; A61L 9/015; F23D 14/32; B01D 53/005; B01D 53/76; F01N 3/36; F23G 7/06; B01D 2251/102; B01D 2258/01; B01D 2258/06; B01D 2257/93; B01D 2259/4508; F23G 2900/7005; B01D 2251/202

(57) ABSTRACT

The invention relates to a device and a method for cleaning a gas in a confined environment, said device comprising a cleaning room with an inlet for the gas to be cleaned, an inlet for a reactant gas for combustion in said cleaning room and an ignition device for combusting said reaction gas, and an outlet for the mixture of cleaned gas and combustion products from the cleaning space. The reactant gas comprises a substantially stoichiometric mixture of H₂ and O₂.

The inlet for the gas to be cleaned may be in contact with the residence environment and the cleaning room, and the outlet for the mixture of cleaned gas and combustion products may be in contact with the cleaning room and the residence environment. The device also comprises a radiation element that is in radiating contact with the residence environment, wherein the radiation element is in heat-exchanging contact with at least one of [a] the combustion flame of the reactant gas and [b] the mixture of cleaned gas and combustion products from the cleaning space.

The method and device may be used for cleaning exhaust gases of a vehicle.

### CROSS-REFERENCE TO RELATED APPLICATIONS

This application is a continuation application of Patent Cooperation Treaty Application No. PCT/NL2016/050483, filed on Jul. 6, 2016, which claims priority to Netherlands Patent Application No. 2015095, filed Jul. 6, 2015, and the specifications and claims thereof are incorporated herein by reference.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### STATEMENT REGARDING PRIOR DISCLOSURES BY THE INVENTOR OR A JOINT INVENTOR

Not Applicable.

### COPYRIGHTED MATERIAL

Not Applicable.

### BACKGROUND OF THE INVENTION

The present invention relates to a device for cleaning a gas according to the preamble of claim 1. The invention also relates to a method according to claim 9 for cleaning a gas by using the device according to the invention.

Such a device is known in the art. For example, it is known to submit a contaminated gas to a high temperature for cleaning same, as may be obtained by contacting said gas with a flame of an organic fuel.

Such known device has drawbacks. When a contaminated gas must be cleaned in a confined area the combustion products, especially but not limited to CO₂, have a detrimental influence on the organisms residing in said area.

Therefore, this known device cannot be used for cleaning gasses in a confined area.

From Chinese patent application CN203349492U a device is known for cleaning a gas. This known device comprises two coaxially positioned cylinders and wherein a flame of HHO and the gas to be cleaned are fed in combination though the inner cylinder. The combustion of hydrogen provides a high temperature such that contaminants are removed. Through the space between said cylinders air is fed so as to prevent the outer cylinder becoming too hot. As a result, a good insulation is obtained.

### BRIEF SUMMARY OF THE INVENTION

The present invention aims at providing an improved device of the kind mentioned in the preamble.

The invention especially aims at providing a device of the kind mentioned in the preamble and that can be used in a confined area where living organisms reside, especially people or animals. The invention therefore aims at providing a device for use in living and stay environments, such as homes and workplaces.

The invention also relates to an improved method of the kind mentioned in the preamble, that is especially suitable for use with the device according to the present invention.

### BRIEF DESCRIPTION OF THE SEVERAL VIEWS OF THE DRAWINGS

Not Applicable.

### DETAILED DESCRIPTION OF THE INVENTION

So as to obtain at least one of the above-mentioned advantages, according to a first embodiment the present invention provides a device comprising the features of claim 1. This device has the advantage that it can be placed in a confined environment for cleaning the air in said environment. For example, the device can be operatively arranged in an confined living or working environment, hereinafter also identified as “room” of “residence area”, wherein the air of said confined area is removed from the device that is placed in said confined area, after which the cleaning operation is performed and after which the cleaned air is returned into said confined area. Due to the absence of CO₂ in the gasses obtained from the cleaning method, due to the absence of organic fuels, the gasses that are returned into the confined area will have no detrimental effect on people or animals residing in said area. As a matter of fact, the term “confined area” also relates to any other room where the gas to be cleaned is removed from, for example a flue pipe, and where the cleaned gas is to be returned to.

Therefore, the device according to the present invention can be used in living rooms, work places, (public) toilets, changing rooms, hospitals, nursing homes, industrial areas like machine rooms, and many other places.

The device can be used favorably for cleaning air that has been contaminated with bacteria and viruses.

The expression “cleaning of a gas” means every way of removing detrimental substances from the gas to be cleaned, like organic substances and non-organic (as well as inorganic) substances. For example, harmful organisms like bacteria and viruses are “detrimental substances”. Examples of organic substances are dust and manure particles and hydrocarbons in substituted and unsubstituted form. An example of an inorganic substance is asbestos that can be converted into a harmless material by means of the method and device according to the present invention. Due to the high combustion temperature of a mixture of H₂ en O₂, which can be more than 2000° C., asbestos loses its original elongated shape such that it can no longer penetrate in human tissue, like lung tissue. Confined areas and spaces that are contaminated with asbestos can in a short time be cleaned from airborne asbestos particles by means of the method and device according to the invention. By applying a fan the advantage is obtained that settled particles, which cannot be removed by any known technique, are re-suspended in the air such that they can be removed by means of the method and device according to the present invention.

In addition to its cleaning function, the device according to the invention is used as a heating element as well. To that end, the radiation element is in radiating contact with the residence environment and in heat-exchanging contact with at least one of [a] the combustion flame of the reactant gas and [b] the mixture of cleaned gas and combustion products from the cleaning space. An efficient heating device is obtained especially when the radiation element comprises a stone or ceramic material. This has a good heat capacity, in combination with an excellent emitting power, so that a pleasant heating is obtained.

An optimum result is obtained when the radiation element serves as outlet from the cleaning room and is porous for passing through the mixture of cleaned gas and combustion products from the cleaning room. Such yields a direct heating of the radiation element, wherein all gasses provide for heating of the element. Such also provides for a diffuse flow of gasses into the room, that is the residence environment.

The device is also suitable for cleaning air that is contaminated with oxidizable matter, for example dust and fine particles, preferably of organic origin. The contaminants will be converted by means of the high combustion temperatures of the hydrogen oxidation. Organic contaminants will be converted completely into oxides of their basis molecules, such that harmless substances remain. The oxygen required for such combustion has to be fed into the cleaning room, together with the air from the surroundings environment that is subjected to the cleaning method.

It has also shown that the present invention greatly improves the air quality in areas that have an air conditioning system. The device according to the invention increases the humidity due to the combustion of hydrogen. Such synergistic improvement was not expected.

Based on the afore it is clear that it is preferred that the inlet of the gas to be cleaned is in direct contact with the residence area and the cleaning room, and wherein the outlet for the mixture of cleaned gas and the combustion products is in contact with the cleaning room and the residence area.

A very efficient mixing of gas to be cleaned and the high temperature reaction gases is obtained when the device comprises a apertured wall for feeding the gas to be cleaned into the reaction room, said apertures (also identified as openings) having a diameter in the range of from 0.1 mm to 3 mm. As a result, the gas to be cleaned is fed to the flame in the reaction room very diffusely and therefore the flame is not influenced due to turbulence of said gas to be cleaned. Thus, the cleaning operation can be performed very efficiently and the amount of reactant gas will be less compared with a method wherein a direct supply of gas is applied.

According to an alternative embodiment the device comprises an apertured wall for guiding there through and out of the cleaning room the gas to be cleaned, said apertures having a diameter in the range of from 0.1 mm to 3 mm. Such provides a good mixture of the gas to be cleaned and the ignited reactant gas, said mixture being obtained substantially inside the apertured wall. The wall will have a high temperature that is substantially identical in the complete wall, such that all gasses to be cleaned will be subjected to the same temperature, even when these gasses do not make direct contact with the flame.

A further development of the present embodiment provides that at least one of [a] the flame of the combusted reactant gas and [b] the mixture of the cleaned gas and combustion products, heats the apertured wall. Thus, the flame can be directly aimed at the wall so as to heat same and wherein the gases to be cleaned that are to be fed through the wall are converted in and near said wall due to the high temperatures at that position. On the other hand, the flame may be directed substantially completely in the cleaning room such that at least a part of the gases to be cleaned will contact the flame inside the cleaning room and wherein the mixture contacts the wall when leaving the cleaning room and thus heats the wall.

It is especially preferred that in the device according to the present invention the radiation element serves as an outlet from the cleaning room and is porous for feeding there through the mixture of cleaned gas and combustion products from the cleaning room. The porous radiation element will preferably be an apertured wall with apertures having a diameter in the range of from 0.1 mm to 3 mm. Such provides a diffuse exit stream of cleaned gas mixture into the room or residence area combined with a pleasant radiation heat.

An advantageous cleaning of the gases is also obtained when the device comprises a cylinder for passing there through part of the gas to be cleaned and the reactant gas combustion flame. By subdividing the gas into a plurality of streams and by feeding each separate stream into a separate cylinder, a relatively low gas speed is yielded compared to an embodiment wherein the gas stream is fd through a single cylinder. Thus, the cleaning is improved substantially. The cylinder preferably has a diameter of maximally twice the flame diameter, yielding an excellent cleaning.

According to a further aspect, the invention relates to a method for cleaning a gas in a confined environment. The method is very suitable for use with the device according to the present invention. Hereinafter, the method will be explained in combination with the device according to the invention. The method comprises the steps of removing from a surrounding area a gas to be cleaned and supplying same to a cleaning room, supplying said reactant gas to the cleaning room and igniting same for combusting the reactant gas, and removing said obtained mixture of cleaned gas and combustion products from the cleaning room, characterized in that the method comprises the step of adding to the cleaning room a substantially stoichiometric mixture of H₂ en O₂ as reactant gas. Thus, the advantages are obtained with the device according to the present invention.

The method can be performed very efficiently by returning the mixture of cleaned gas and combustion products from the cleaning room into the surrounding area. By using hydrogen as fuel and a stoichiometric addition of oxygen into the cleaning room , no oxygen is removed from the environmental area itself, providing a very safe method for cleaning air.

As a consequence, the method itself can be performed even in a closed or confined space.

An efficient mixing of gas mixture from the cleaning room is obtained by a method comprising the step of removing the mixture of cleaned gas and combustion products from the cleaning room through an apertured wall. By positioning this wall in direct contact with the room a very diffuse removal of the gas mixture is obtained. Such provides a great comfort to people resent in the said room.

Finally, it is preferred when applying the method for cleaning exhaust gases of a vehicle, that the method comprises the steps of supplying an exhaust gas into the cleaning room though the inlet, wherein the cleaning room is comprised of a muffler of an exhaust system of a vehicle. Then, a very good cleaning of exhaust gases is obtained, especially with regard to dust particles, generally known as fine dust particles that are efficiently converted and wherein carbon monoxide is converted into CO₂.

According to a further aspect, the invention therefore also relates to a device as mentioned above and that is embodied for cleaning a gas from a combustion engine, for example in a vehicle or a vessel. More in particular, but not exclusively, the invention relates to cleaning of gasses in an exhaust pipe of a vehicle, wherein the cleaning room is provided in a muffler of the exhaust system. The present invention surprisingly provides a suitable method for removing contaminants from exhaust gases. By performing the combustion in the muffler, a surprisingly good cleaning is obtained. When performing said cleaning in a muffler, the cleaning is surprisingly much better than when the combustion, using an identical amount of hydrogen gas and oxygen gas, in regular piping of an exhaust system. By incorporating the device according to the present invention in an exhaust system the contaminants are removed and the humidity is increased. Such leads to a strong relative cleaning of exhaust gases.

The invention is also suitable for the cleaning of combustion gases from fuel oil, such as those released from ships in which the combustion is frequently incomplete, and wherein many incompletely combusted fuel ends up in the flue gases. By positioning the device according to the invention in the flue gas discharge duct, the impurities are removed, and the moisture content is increased, which also here leads to a strong relative cleaning of the flue gases.

When burning exhaust gases and flue gases it is preferred that the device has a cylinder for guiding through part of the gas to be cleaned a well as the combustion flame of the reactant gas. By dividing the gas feed in a plurality of streams and feeding each stream into a separate cylinder, for example in a muffler of an exhaust system or in a flue duct, a relatively lower gas speed is obtained compared to a situation where the complete gas stream is fed through a single of such cylinder. The cleaning is improved significantly. The cylinder preferably has a diameter that is maximally twice the diameter of the flame, providing an excellent cleaning.

According to another aspect, at least part of the HHO that is available can be added to the combustion chamber of the engine so as to add to a cleaner and more complete combustion of fuel in said engine of the vehicle or vessel.

### Example I

### Cleaning of Exhaust Gases

The device used in this example is incorporated in an exhaust system of a vehicle, wherein a single HHO generator is positioned separately in said vehicle. The HHO generator provides hydrogen gas and oxygen gas in a stoichiometric amount as a gas mixture (referred to as HHO). The yield in continuous production of this HHO generator is 1 lpm of HHO.

The vehicle used is a passenger car of made FAW, type N5 (manufactured 2013), with a petrol engine.

The HHO was combusted in a total of three flames in an open part of the muffler of the exhaust system. All exhaust gases were fed through the cleaning room in this muffler. The muffler was subdivided in a few compartments and provided with damping material, as is usual for a muffler.

The vehicle was provided with a catalyst. Without using HHO (but while using the catalyst) The concentration of carbon monoxide CO amounted to 450 ppm.

The result of carbon monoxide CO when using the HHO cleaning according to the present invention was less than 200 ppm. The HHO device therefore provides an additional reduction of carbon monoxide CO of more than 55%, even after the exhaust gases had been treated by the catalyst.

Positioning the cylinder around the flame, from a position around the burner and extending towards a position a few centimeters downstream of the exit of the burner, provides a clearly more stable flame. The rate of reduction of CO is not influenced by this measure.

### Example II

### Cleaning of Room Air

The device according to the invention is embodied as a container comprising a water vessel, an HHO generator for providing a stoichiometric amount of hydrogen and oxygen (HHO) including control electronics, a fan for removing air from the room and into the device and returning said treated air back into the room. The device also comprises a series of burners for combustion HHO in a controlled manner.

An HHO generator producing an amount of HHO of for example 4 lpm can be suitably used for cleaning air in a room of for example 40 to 100 m3. However, in case many contaminants are present the amount of HHO can be increased.

When using the device according to the invention, an feed of cleaned air into the room is obtained. Small particles have been removed from this air stream. Contaminants like bacteria and viruses are removed as well effectively. Therefore, the device according to the present invention is suitable for performing the method according to the present invention, for example in hospitals where outbreak of contagious illnesses and bacterial infections can be effectively prevented.

The invention is not limited to the embodiments specifically described above.

For example, a fan can be provided for forcing gasses to be cleaned through the device according to the invention. In case of an air cleaner, such fan may have higher usefulness in an air cleaner than in a device for cleaning exhaust gases. Especially when using a porous piece of material through which the gases that are obtained in the combustion process are to be forced, a fan may be used advantageously.

The invention is applicable in all kinds of internal combustion engines, such as petrol, diesel and marine diesel engines. Due to the application of the device and method of the present invention, a depletion of unburned fuels is always obtained, among which nitrogen monoxide and carbon monoxide as well as unburnt hydrocarbons.

When cleaning air from the surrounding, for example outside air that is added to a residence area, or air that is retrieved from a residence area and recirculated into said residence area, an optimal cleaning is obtained. Both bacteria and viruses and other germs are destroyed by means of the method according to the present invention and in the device according to the present invention. Especially when used in combination with a porous plate for removing combustion products from the cleaning room a complete cleaning of such harmful organisms is obtained.

A particular application is found in hospitals and other places where infected people are nursed and treated. The number of harmful organisms in the treated air is null, strongly contributing to the reduction of spreading of bacteria and viruses. As a consequence, the presence and spread of MRSA and NDM-1 bacteria can be reduced to null.

The method and device according to the present invention can be applied profitably in livestock farming. In particular, in extensive livestock, such as poultry including chickens, pigs, and cows, in particular those where livestock remains inside the farms the whole year, a clean air is of great importance. The present invention can easily provide for a germ-free air that is supplied to the sheds. As a consequence, the amount of antibiotics to be administered to the livestock can be greatly reduced. The chance of resistant bacteria is thus greatly reduced, adding to public health.

In the examples and in the general description of the invention, it is assumed that the HHO gas is produced as a mixture and is supplied as a mixture to the burners. Of course, the mixture of H₂ and O₂ gas produced by the HHO generator may be collected separately and fed separately to the burner(s).

The invention is limited only by the appended claims.

The invention also relates to any combination of features that have been described above independently from each other.

What is claimed is:

1. A device for cleaning a gas in a confined environment, said device comprising a cleaning room with an inlet from a residence environment for the gas to be cleaned, an inlet for a reactant gas for combustion in said cleaning room and an ignition device for combusting said reaction gas, and an outlet for the mixture of cleaned gas and combustion products from the cleaning space, wherein the reactant gas comprises a substantially stoichiometric mixture of H₂ and O₂; wherein the inlet for the gas to be cleaned is in contact with the residence environment and the cleaning room, and wherein the outlet for the mixture of cleaned gas and combustion products is in contact with the cleaning room and the residence environment;
characterized in that:
the device comprises a radiation element that is in radiating contact with the residence environment, wherein the radiation element is in heat-exchanging contact with at least one of [a] the combustion flame of the reactant gas and [b] the mixture of cleaned gas and combustion products from the cleaning space.

2. A device according to claim 1, comprising an apertured wall for guiding there through the gas to be cleaned into the cleaning room, said apertures having a diameter in the range of from 0.1 mm to 3 mm.

3. A device according to claim 1, comprising an apertured wall for guiding there through and out of the cleaning room the cleaned gas and combustion products, said apertures having a diameter in the range of from 0.1 mm to 3 mm.

4. A device according to claim 3, wherein at least one of [a] the flame of the combusted reactant gas and [b] the mixture of the cleaned gas and combustion products heats the apertured wall.

5. A device according to claim 1, wherein the radiation element comprises a stone or ceramic material.

6. A device according to claim 1, wherein the radiation element serves as an outlet from the cleaning room and is porous for the passage of the mixture of cleaned gas and combustion products from the cleaning room, and wherein preferably the radiation element comprises an apertured wall with apertures with a diameter in the range of from 0.1 mm to 3 mm.

7. A device according to claim 1, comprising a cylinder for passing there through the gas to be cleaned and the reactant gas combustion flame.

8. A device according to claim 1 for cleaning a gas in a confined environment, said device comprising a cleaning room with an inlet for the gas to be cleaned, an inlet for a reactant gas for combustion in the cleaning room with an ignition for combustion of the reactant gas, and an outlet for the mixture of cleaned gas and combustion products from the cleaning room, characterized in that the cleaning room comprises an inlet for a reactant gas that comprises a substantial stoichiometric mixture of H₂ en O₂, said device being embodied for cleaning a gas in an exhaust pipe system of a vehicle, wherein the cleaning room is provided in the muffler of the exhaust pipe system.

9. A method for cleaning a gas in a confined environment, said method comprising the steps of removing from a surrounding area a gas to be cleaned and supplying same to a cleaning room, supplying said reactant gas to the cleaning room and igniting same for combusting the reactant gas, said reactant gas substantially being a stoichiometric mixture of H₂ en O₂, and removing said obtained mixture of cleaned gas and combustion products from the cleaning room and supplying said mixture to the surrounding area, characterized in that the method comprises the step of emitting radiation heat to said surrounding area by means of a radiation element, said radiation element being on the one hand in radiating contact with the surrounding area and on the other hand in heat exchanging contact with at least one of [a] the combustion flame of the reactant gas and [b] the mixture of the cleaned gas and the combustion products from the cleaning room.

10. A method according to claim 9, wherein the mixture of cleaned gas and combustion products from the cleaning room is returned into the surrounding area.

11. A method according to claim 10, wherein the surrounding area is a confined environment.

12. A method according to claim 9, comprising the step of removing the mixture of cleaned gas and combustion products from the cleaning room through an apertured wall.

13. A method according to claim 9, comprising the step of supplying an exhaust gas into the cleaning room though the inlet, wherein the cleaning room is comprised of a muffler of an exhaust system of a vehicle.

14. A method according to claim 9, wherein the gas to be cleaned is asbestos comprising air.

15. A method according to claim 14, wherein the mixture of cleaned gas and combustion products is returned into the confined environment.
